(19) United States
(12) Patent Application Publication
(10) Pub. No.: US 2026/0101427 A1
(43) Pub. Date: Apr. 9, 2026

(54) SYSTEMS AND METHODS FOR ADAPTIVELY CONTROLLING FILAMENT CURRENT IN AN X-RAY TUBE

(71) Applicant: Hologic, Inc., Marlborough, MA (US)

(72) Inventors: Guoyun Ru, Farmington, CT (US); David Aizer, Danbury, CT (US); J. Austin Fraley, Danbury, CT (US); Edward Nonnweiler, Brookfield, CT (US)

(73) Assignee: Hologic, Inc., Marlborough, MA (US)

(21) Appl. No.: 19/298,478

(22) Filed: Aug. 13, 2025

Related U.S. Application Data
(63) Continuation of application No. 18/100,683, filed on Jan. 24, 2023, now Pat. No. 12,414,217.
(60) Provisional application No. 63/307,311, filed on Feb. 7, 2022.

(51) Int. Cl. H05G 1/34; A61B 6/50; A61B 6/58; H05G 1/26; H05G 1/54

(52) U.S. Cl. CPC H05G 1/34; A61B 6/582; H05G 1/265; H05G 1/54; A61B 6/502

(57) ABSTRACT

Systems and methods of adaptively controlling filament current in an x-ray tube of an imaging system include the x-ray tube having a filament being calibrated. Calibration data from the calibration of the x-ray tube is stored at the imaging system, the calibration data including a filament current value that determines a tube current value for a tube voltage value at a plurality of stations. A resistance value of the filament over a period of time is monitored. A change in the resistance value of the filament over the period of time is determined, and the filament current value of at least one of the plurality of stations is adjusted based on the changed resistance value.

### CROSS-REFERENCE TO RELATED APPLICATIONS

This application is a continuation of U.S. patent application Ser. No. 18/100,683, filed Jan. 24, 2023, which claims the benefit of U.S. Provisional Application No. 63/307,311, filed Feb. 7, 2022, the entire disclosures of which are incorporated herein by reference in their entireties. To the extent appropriate, a claim of priority is made to each of the above disclosed applications.

### BACKGROUND

Imaging based on the use of x-rays is commonplace in medical imaging technology, such as, but not limited to, mammography or tomosynthesis systems. The x-rays used in such imaging technology are often generated through the use of an x-ray tube. Inside the x-ray tube there is an anode and a cathode, and within the cathode there is a filament that emits electrons. The electrons are accelerated by an electrical field generated by applying a high voltage potential across the anode and cathode terminals. As the filament wears with use, the filament material evaporates under high temperature. This evaporation results in a thinning of the material and an increase in filament resistance. When the resistance of the filament increases, power and temperature at the filament are increased, thereby resulting in an increased radiation output.

### SUMMARY

In one aspect, the technology relates to a method of adaptively controlling filament current in an x-ray tube of an imaging system, the method including: calibrating the x-ray tube having a filament; storing calibration data from the calibration of the x-ray tube at the imaging system, wherein the calibration data includes a filament current value that determines a tube current value for a tube voltage value at a plurality of stations; monitoring a resistance value of the filament over a period of time; determining a change in the resistance value of the filament over the period of time; and adjusting the filament current value of at least one of the plurality of stations based on the changed resistance value.

In an example, monitoring the resistance value of the filament over the period of time includes measuring the resistance value after a predetermined number of sequence exposures and storing each measured resistance value. In another example, measuring the resistance value after the predetermined number of sequence exposures includes applying a constant filament current value to the filament after each of the predetermined number of sequence exposures. In yet another example, the method further includes waiting a predetermined time period between an end of the predetermined number of sequence exposures and prior to applying the constant filament current value to the filament for resistance value measurement. In still another example, the filament current value of each of the plurality of station is updated based on the changed resistance value. In an example, determining the change in the resistance value includes comparing a difference between resistance values over the period of time to a predetermined benchmark.

In another example, the method further includes measuring a resulted tube current value from the x-ray tube at a sequence exposure having a stored tube current value and tube voltage value for a station of the plurality of stations; and comparing a difference between the measured resulted tube current value and the stored tube current value for the respective station. In yet another example, based on the difference between the measured resulted tube current value and the stored tube current value, the period of time is at least partially defined. In still another example, measuring resulted tube current value is performed pre-exposure or post-exposure of the sequence exposure. In an example, the method further includes determining a life-cycle period of the filament based at least partially on the monitored resistance value of the filament.

In another aspect, the technology relates to an imaging system includes: an x-ray tube having a filament; a current control circuit coupled to the x-ray tube and configured to channel current through the filament; at least one processor communicatively coupled to the current control circuit; and memory communicatively coupled to the at least one processor, the memory including computer executable instructions that, when executed by the at least one processor, performs a method including: calibrating the x-ray tube; storing calibration data from the calibration of the x-ray tube at the imaging system, wherein the calibration data includes a filament current value that determines a tube current value for a tube voltage value at a plurality of stations; monitoring a resistance value of the filament over a period of time; determining a change in the resistance value of the filament over the period of time; and adjusting the filament current value of at least one of the plurality of stations based on the changed resistance value.

In an example, monitoring the resistance value of the filament over the period of time includes measuring the resistance value after a predetermined number of sequence exposures and storing each measured resistance value. In another example, measuring the resistance value after the predetermined number of sequence exposures includes applying a constant filament current value to the filament after each of the predetermined number of sequence exposures. In yet another example, the method further includes waiting a predetermined time period between an end of the predetermined number of sequence exposures and prior to applying the constant filament current value to the filament for resistance value measurement. In still another example, the filament current value of each of the plurality of station is updated based on the changed resistance value. In an example, determining the change in the resistance value includes comparing a difference between resistance values over the period of time to a predetermined benchmark.

In another example, the method further includes: measuring a resulted tube current value from the x-ray tube at a sequence exposure having a stored tube current value and tube voltage value for a station of the plurality of stations; and comparing a difference between the measured resulted tube current value and the stored tube current value for the respective station. In yet another example, based on the difference between the measured resulted tube current value and the stored tube current value, the period of time is at least partially defined. In still another example, measuring resulted tube current value is performed pre-exposure or post-exposure of the sequence exposure. In an example, the method further includes determining a life-cycle period of the filament based at least partially on the monitored resistance value of the filament.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

FIG. 1 is a schematic view of an exemplary imaging system.

FIG. 2 is a perspective view of the imaging system of FIG. 1.

FIG. 3 is a schematic view of an x-ray tube.

FIG. 4 is a graph depicting filament resistance change over a life of an x-ray tube.

FIG. 5 is a graph depicting filament current change over the life of an x-ray tube.

FIG. 6 is a graph depicting filament power change over the life of an x-ray tube.

FIG. 7 depicts a flowchart illustrating a method of adaptively controlling filament current in an x-ray tube.

FIG. 8 depicts an example of a suitable operating environment for use with the present examples.

### DETAILED DESCRIPTION

As discussed above, x-ray tubes in medical imaging systems have limited lifetimes. The limited lifetime of x-ray tubes is often due to the high heat and high voltages that are generally required for the operation of the x-ray tube. The high heat and voltages cause the components of the x-ray tube to break down, and in some components, change performance characteristics. When the x-ray tube changes performance characteristics, the x-ray tube needs to be recalibrated, and in some instances replaced. Recalibration and/or replacement costs of the x-ray tube are often significant, and according, improvements to the x-ray tube are desired.

Based on analysis of x-ray tube filaments, as the filament degrades with use, the filament material evaporates and reduces its thickness. This results in the resistive properties of the filament changing and increasing. As the resistance of the filament increases, the power and the temperature generated at the filament increases during operation, causing an increase in radiation output from the x-ray tube.

The present technology relates to a feedback mechanism to adjust the filament current needed to maintain the required or desired x-ray radiation at each tube output current and tube voltage station as the filament resistance changes over time. For example, after calibration of the x-ray tube, the filament power (e.g., based on the filament current generated) for each tube output current and tube voltage station is calculated and saved by the control system. Over the operational life of the x-ray tube, the filament resistance is monitored. When the filament resistance changes by a predetermined amount, the filament current may be adjusted appropriately by the control system to get the filament power the same as the stored value for each tube output current and tube voltage station. As such, the x-ray tube can deliver the desired x-ray radiation even with the filament changing resistive characteristics. Additionally, recalibration procedures will be reduced as the x-ray tube degrades with use.

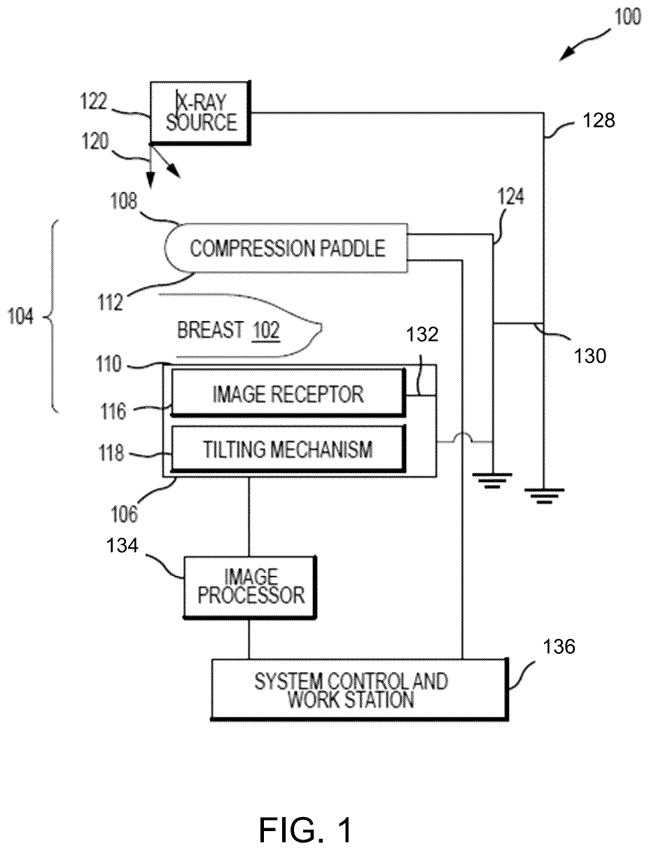

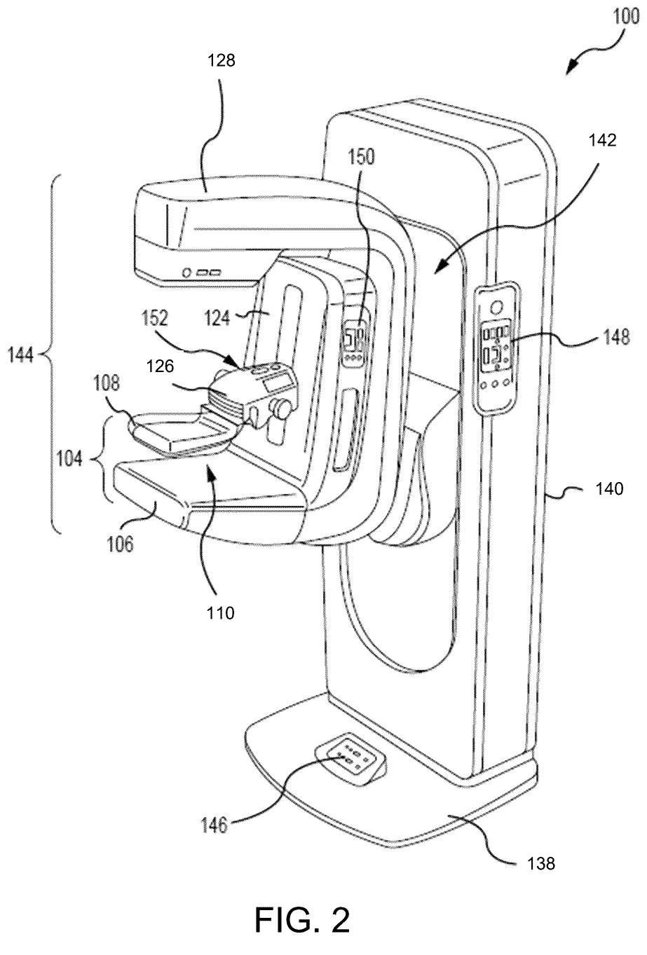

FIG. 1 is a schematic view of an exemplary imaging system 100. FIG. 2 is a perspective view of the imaging system 100. Referring concurrently to FIGS. 1 and 2, not every element described below is depicted in both figures. The imaging system 100 immobilizes a patient's breast 102 for x-ray imaging (either or both of mammography, tomosynthesis, or other imaging modalities) via a breast compression immobilizer unit 104 that includes a breast support platform 106 and a moveable compression paddle 108. The breast support platform 106 and the compression paddle 108 each have a compression surface 110 and 112, respectively, that move towards each other to compress, immobilize, stabilize, or otherwise hold and secure the breast 102 during imaging procedures. In known systems, the compression surface 110, 112 is exposed so as to directly contact the breast 102. The platform 106 also houses an image receptor 116 and, optionally, a tilting mechanism 118, and optionally an anti-scatter grid (not depicted, but disposed above the image receptor 116). The immobilizer unit 104 is in a path of an imaging beam 120 emanating from x-ray source 122, such that the beam 120 impinges on the image receptor 116.

The immobilizer unit 104 is supported on a first support arm 124 and the compression paddle 108 is supported on the first support arm 124 by a paddle mount 126, which is configured to be raised and lowered along the support arm 124. The x-ray source 122 is supported on a second support arm, also referred to as a tube head 128. For mammography, support arms 124 and 128 can rotate as a unit about an axis 130 between different imaging orientations such as CC and MLO, so that the system 100 can take a mammogram projection image at each orientation. In operation, the image receptor 116 remains in place relative to the platform 106 while an image is taken. The immobilizer unit 104 releases the breast 102 for movement of arms 124, 128 to a different imaging orientation. For tomosynthesis, the support arm 124 stays in place, with the breast 102 immobilized and remaining in place, while at least the second support arm 128 rotates the x-ray source 122 relative to the immobilizer unit 104 and the compressed breast 102 about the axis 130. The system 100 takes plural tomosynthesis projection images of the breast 102 at respective angles of the beam 120 relative to the breast 102.

Concurrently and optionally, the image receptor 116 may be tilted relative to the breast support platform 106 and in sync with the rotation of the second support arm 128. The tilting can be through the same angle as the rotation of the x-ray source 122, but may also be through a different angle selected such that the beam 120 remains substantially in the same position on the image receptor 116 for each of the plural images. The tilting can be about an axis 132, which can but need not be in the image plane of the image receptor 116. The tilting mechanism 118 that is coupled to the image receptor 116 can drive the image receptor 116 in a tilting motion. For tomosynthesis imaging and/or CT imaging, the breast support platform 106 can be horizontal or can be at an angle to the horizontal, e.g., at an orientation similar to that for conventional MLO imaging in mammography. The system 100 can be solely a mammography system, a CT system, or solely a tomosynthesis system, other modalities such as ultrasound, or a “combo” system that can perform multiple forms of imaging. An example of a system has been offered by the assignee hereof under the trade name Selenia Dimensions.

Whether operating in a mammography or a tomosynthesis mode, the system 100 images the breast 102 by emitting the x-ray beam 120 from the x-ray source 122. The x-ray beam 120 passes through the breast 102 where it is detected by the image receptor 116. The image receptor 116 may include a plurality of pixels that detect the intensity of the x-ray beam 120 at a plurality of locations after the x-ray beam 120 has passed through the breast 102. The attenuation of the x-ray beam 120 as it passes through the breast 102 changes depending on the structures of the breast 102. Accordingly, images of the breast 102 may be produced from the detected x-ray beam 120. For instance, the image receptor 116 produces imaging information in the form of electric signals, and supplies that imaging information to an image processor 134 for processing and generating x-ray images of the breast 102. A system control and work station unit 136 including software controls the operation of the system 100 and interacts with the operator to receive commands and deliver information including processed-ray images. The system control and work station unit 136 may also include software for controlling the operation of the x-ray source 122.

The imaging system 100 includes a floor mount or base 138 for supporting the imaging system 100 on a floor. A gantry 140 extends upwards from the floor mount 138 and rotatably supports both the tube head 128 and the support arm 124. The tube head 128 and support arm 124 are configured to rotate discretely from each other and may also be raised and lowered along a face 142 of the gantry 140 so as to accommodate patients of different heights. The x-ray source 122 is disposed within the tube head 128. Together, the tube head 128 and support arm 124 may be referred to as a C-arm 144. A number of interfaces and display screens are disposed on the imaging system 100. These include a foot display screen 146, a gantry interface 148, a support arm interface 150, and a compression arm interface 152. In general the various interfaces 146, 148, 150, and 152 may include one or more tactile buttons, knobs, switches, as well as one or more display screens, including capacitive touch screens with graphic user interfaces (GUIs) so as to enable user interaction with and control of the imaging system 100.

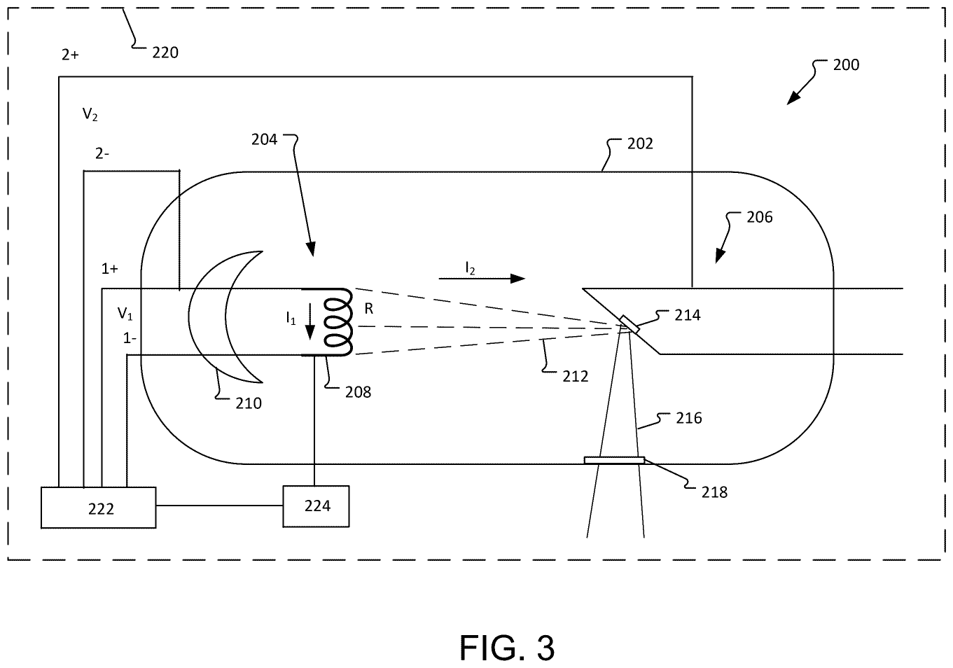

FIG. 3 is a schematic view of an x-ray tube 200. The x-ray tube 200 may be included as at least part of the x-ray source 122 discussed above. The x-ray tube 200 includes a tube body 202 housing a cathode 204 and an anode 206. The cathode 204 includes a filament 208 and, in some examples, a focusing cup 210. The filament 208 can be placed adjacent the focusing cup 210 and between the focusing cup 210 and the anode 206. The filament 208 may be formed from a material with a high melting point, such as tungsten. A voltage or signal V₁ may be applied across the filament 208 via wires connected to each end of the filament 208, indicated by the 1+ for the positive connection to the filament 208 and the 1− for the negative connection to the filament 208. When the signal or voltage is applied across the filament 208, a current I₁ flows through the filament 208 which heats the filament 208 and causes electrons to be emitted from the filament 208. Due to a high voltage potential V₂ applied between the cathode 204 and the anode 206 as indicated by the 2+ for the positive connection to the anode 206 and the 2− for the negative connection to the cathode 204, the electrons emitted from the filament 208 are accelerated towards the anode 206. The accelerated electrons form an electron beam 212 that travels along an electron beam path between the cathode 204 and the anode 206. The electron beam 212 impacts the anode 206 at a focal spot 214 and causes the emission of x-rays 216 from the anode 206. The x-rays 216 exit the x-ray tube body 202 through a tube window 218. The x-rays 216 that exit from the body 202 form the x-ray beam that is used for imaging, such as the x-ray beam 120 discussed above with reference to FIGS. 1 and 2.

The area that the electron beam 212 impacts the anode 206 is referred to as the focal spot 214. This size of the focal spot 214 relates to the resolution required or desired for the imaging process. The location of the focal spot 214 on the anode 206, as well as, the angle of the anode 206, have an effect on the direction of the x-rays 216 produced from the anode 206. The size and location of the focal spot 214 may be controlled or modified by the focusing cup 210. Additionally, the electron beam 212 is produced by thermionic effect from the filament 208 being heated by the electric current I₁. This current value of the filament 208, via applied voltage V₁ across the filament 208, determines a tube output value I₂, typically measured in milliamperes (mA), for a given tube high voltage potential V₂, typically measured in kilovoltage (kV).

In the example, the x-ray tube 200 is included in a high voltage control circuit 220 (also known as a current control circuit) that is configured to control operation of the x-ray tube 200. The high voltage control circuit 220 may include a micro-computer 222 that stores operational data and processes for the x-ray tube 200 and facilitates operation of the x-ray tube 200. For example, the high voltage control circuit 220, via the micro-computer 222 coupled in communication with the x-ray tube 200, applies voltage V₁ across the filament 208 (e.g. via channeling current through the filament) based on stored calibration data so as to emit x-rays 216 having a required or desired radiation amount (e.g., radiation dose). In x-ray imaging, control of the amount of x-ray radiation for increased image quality is desired. In an aspect, the x-ray dose is proportional to the tube output value I₂ per second (mAs) for a given kV tube voltage potential V₂.

In operation, when a new x-ray tube 200 is installed in an x-ray imaging system, the current applied II (e.g., via voltage V₁ generation) at the filament 208 is calibrated for a plurality of kV tube voltage V₂ and mA tube output I₂ stations. In an aspect, every x-ray tube 200 can have unique performance characteristics, and as such, each x-ray tube may have a slightly different calibration. Additionally, the x-ray tube 200 can also be recalibrated as required or desired as performance characteristics change. As used herein, a station corresponds to discrete kV tube voltage V₂ and mA tube output I₂ values that produce, and reproduce, the same quantity of radiation during an exposure. In an aspect, power applied at the filament 208 is based at least partially on filament current I₁ and controls the tube output current values. However, as the filament 208 wears and degrades with use, the filament material evaporates under high temperature, which results in a thinning of the filament material and in turn, an increase of filament resistance R. As the resistance R increases in the filament 208, during operation of the x-ray tube, the power (e.g., via the voltage V₁ applied) generated at the filament 208 increases and the temperature of the filament 208 increases, thereby resulting in an increased radiation output from the same voltage V₁ being applied across the filament 208.

In some systems, the high voltage control circuit 220 may measure the mA tube output I₂ of the x-ray tube after every exposure sequence (e.g. via an ammeter or the like). As used herein, an exposure sequence is the x-ray emissions during a modality procedure. Accordingly, in some modalities, such as mammography, an exposure sequence may be a single emission and the mA tube output I₂ measurement occurs prior to firing the x-ray. In other modalities, such as tomosynthesis, an exposure sequence may be a plurality of emissions and the mA tube output I₂ measurement occurs after firing the x-rays. This measured actual mA output value I₂ of the exposure sequence may then be compared to the desired mA output value I₂ for the exposure sequence. If the difference between the actual mA output value I₂ and the desired mA output value I₂ exceeds a predetermined threshold, then the control circuit 220 may prompt for a new filament calibration. In an aspect, the predetermined threshold may be based on how much different the actual output is from the desired output while still maintaining accurate and usable x-ray images. In some examples, the predetermined threshold may be between about 35%-20%. In other examples, the predetermined threshold may be about 33%. However, if the difference between the actual mA output value I₂ and the desired mA output value I₂ does not exceed the predetermined threshold, then the control circuit 220 can adjust the voltage V₁ being applied to the filament 208 for the next exposure sequence. This voltage adjustment by the control circuit 220 enables the subsequent measured mA output values I₂ to be closer to the desired mA output values I₂ and increase x-ray imaging performance.

Adjusting the voltage V₁ applied to the filament 208 in the process described above, however, only adjusts the voltage V₁ for the active kV tube voltage V₂ and mA tube output I₂ station relating to the specific the exposure sequence. The other stations (e.g., inactive stations) that are not in use do not gain the benefit of the voltage adjustment algorithm. Furthermore, the mA tube output I₂ measurements only account for the fact that the mA output has changed in the x-ray tube 200 and does not address the underlying tube characteristics that caused the changed mA output, and as such, the accuracy of the adjustments are reduced. Accordingly and as described above, it is the resistive properties R of the filament 208 that change over time and result in the changing mA output values I₂ of the x-ray tube 200. Thus, and in the examples described herein, the high voltage control circuit 220 is configured to monitor the resistive properties R of the filament 208. As such, the current In applied to the filament 208 (e.g., via voltage V₁) may be adjusted based on the monitoring of the resistance properties R of the filament 208. By using the resistive properties R of the filament 208, each and every k V tube voltage V₂ and mA tube output I₂ station can be adjusted, thereby increasing x-ray imaging performance. Additionally, the accuracy of the adjustments from the x-ray tube calibration baseline values are increased because the adjustments are based on the underlying changing characteristics of the filament 208 that cause the change in mA output values I₂.

In the example, the high voltage control circuit 220 can include a monitoring instrument 224 operatively controlled by the micro-computer 222 to monitor the resistance values R of the filament 208. In an aspect, the monitoring instrument 224 may be a multimeter or the like that is configured to measure electrical properties of the filament 208, such as one or more of voltage, resistance, and current. Additionally or alternatively, the monitoring instrument 224 can calculate resistance R of the filament 208 using Ohm's law when the current and voltage applied across the filament 208 are known. The control circuit 220 also may be configured with a clock or other time keeping instrument whereby elapsed time may be monitored and certain operations can be performed at predetermined time sampling periods, intervals, or cycles. Accordingly, when the resistance R of the filament 208 changes by a certain amount over time, an algorithm for the control circuit 220 can adjust the filament current I₁, via the voltage V₁, to get the filament power the same as the stored calibration value for each kV tube voltage V₂ and mA tube output I₂ station. This leads to the x-ray tube 200 being capable of delivering the required or desired mA tube output value I₂ even with changing filament resistance values R.

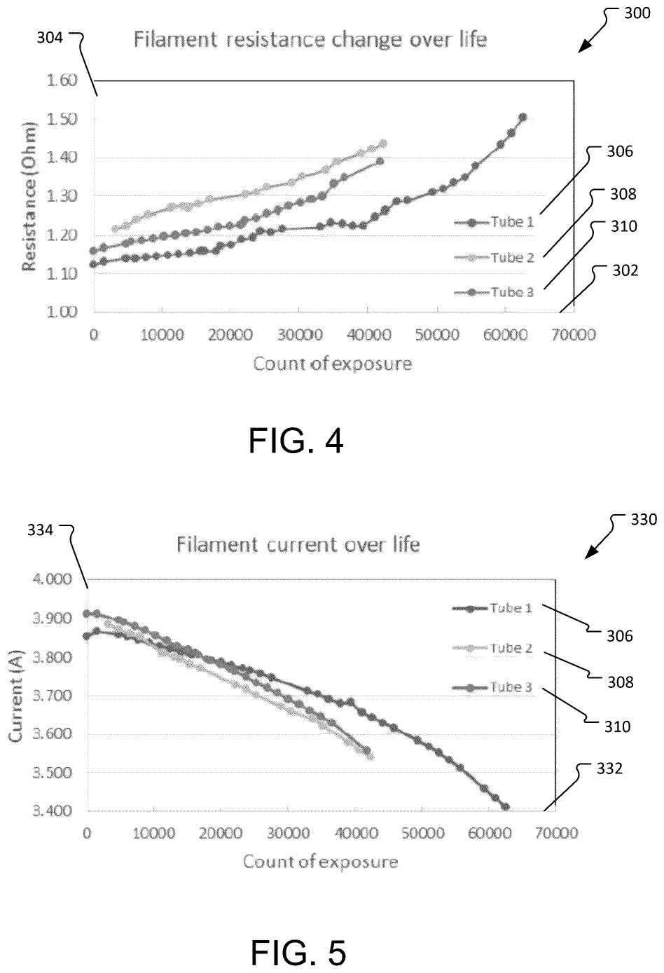

FIG. 4 is a graph 300 depicting filament resistance change over a life of the x-ray tube 200 (shown in FIG. 3). As described above, the change in the resistance properties of the filament 208 (shown in FIG. 3) changes over the operational life of the x-ray tube 200 due to evaporation of filament material under high temperatures. The graph 300 has an x-axis 302 that counts the number of exposures of the x-ray tube 200, and thus, is a time component for the filament 208. In the example, the x-ray exposures are at a 33 kV tube voltage and a 200 mA tube current with 60 mAs. The mAs is the product of tube current in mA and time in seconds. A y-axis 304 charts the resistance in Ohms of the filament 208. As illustrated in the graph 300, three different x-ray tubes 306, 308, 310 are shown to have their filament resistance change during the operational use of the x-ray tubes. Each x-ray tube 306, 308, 310 is slightly different because all x-ray tubes are unique due to the design and manufacture of the individual x-ray tubes. However, all of the x-ray tubes 306, 308, 310 demonstrate that filament resistance values generally increase over time. This time period of change is over a period ten-thousand plus exposure counts, and thus, filament resistance does not change noticeably after every exposure count.

As used herein, the term “life” or “life-cycle” of the x-ray tube does not necessarily mean the life cycle to a point of physical failure. Rather, “life” or “life cycle” refers to a period of usage after which the x-ray tube no longer performs as required or desired.

FIG. 5 is a graph 330 depicting filament current change over the life of the x-ray tube 200 (shown in FIG. 3). As discussed in reference to FIG. 4 above, the resistance properties of the filament 208 (shown in FIG. 3) changes during operational use of the x-ray tube 200. Accordingly, as the filament resistance changes, the current applied at the filament 208 has to be changed during the operational use of the x-ray tube 200 to get desired tube output. The graph 330 has an x-axis 332 that counts the number of exposures of the x-ray tube 200, and thus, is a time component for the filament 208. A y-axis 334 charts the current in amperes of the filament 208. The graph 330 shows the filament current of the same three x-ray tubes 306, 308, 310 as FIG. 4 and at 33 kV tube voltage and 200 mA tube output. The curves illustrate the change in the filament current needed to maintain a constant 200 mA tube output as the filament resistance value increases over time.

Typically, when a new x-ray tube is installed in an imaging system, the filament current is calibrated for the required or desired kV tube voltage and mA tube current stations and these calibration values are stored within the system so that imaging can occur with the correct amount of x-ray radiation. This calibration process also occurs during recalibration of the x-ray tube 200. However, as shown in the graphs 300, 330, the resistive properties of the filament 208 changes and the filament current being applied at the filament 208 has to be reduced in order to deliver the required or desired mA tube output value. This is because as the filament resistance rises, the power at the filament 208 increases (e.g., power P=I₂×R) and the temperature of the filament increases if filament current is not adjusted, resulting in increased radiation output at the x-ray tube 200. As described herein, monitoring the change in resistance values of the filament 208 and generating feedback control of the x-ray tube 200 based on the filament resistance allows the filament current to be adjusted to maintain a required or desired x-ray radiation at each kV tube voltage and mA tube output station.

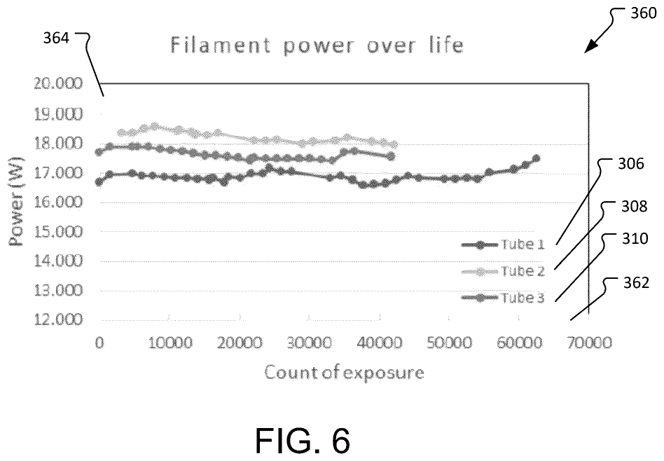

FIG. 6 is a graph 360 depicting filament power change over the life of the x-ray tube 200 (shown in FIG. 3). The graph 360 has an x-axis 362 that counts the number of exposures of the x-ray tube 200, and thus, is a time component for the filament 208 (shown in FIG. 3). A y-axis 364 charts the power in watts of the filament 208. The graph 360 shows the filament power of the same three x-ray tubes 306, 308, 310 as FIGS. 4 and 5, and at a 33 kV tube voltage and 200 mA tube output. These curves demonstrate that to maintain the same mA tube output over the life of the x-ray tube 200 it is required to maintain the same filament power as right after filament current calibration for each kV tube voltage and mA tube output station. As such, to keep mA tube output within a required or desired value with filament resistance increasing over time, the filament current must be adjusted periodically to operate at a proper filament current for each station.

Referring to FIGS. 4-6, the graphs illustrate that filament current adjustment over life of the filament is needed to get a required or desired tube current mA. While FIGS. 4-6 are illustrative of a single station, the algorithm described herein can adjust filament current across active and inactive stations within the imaging system.

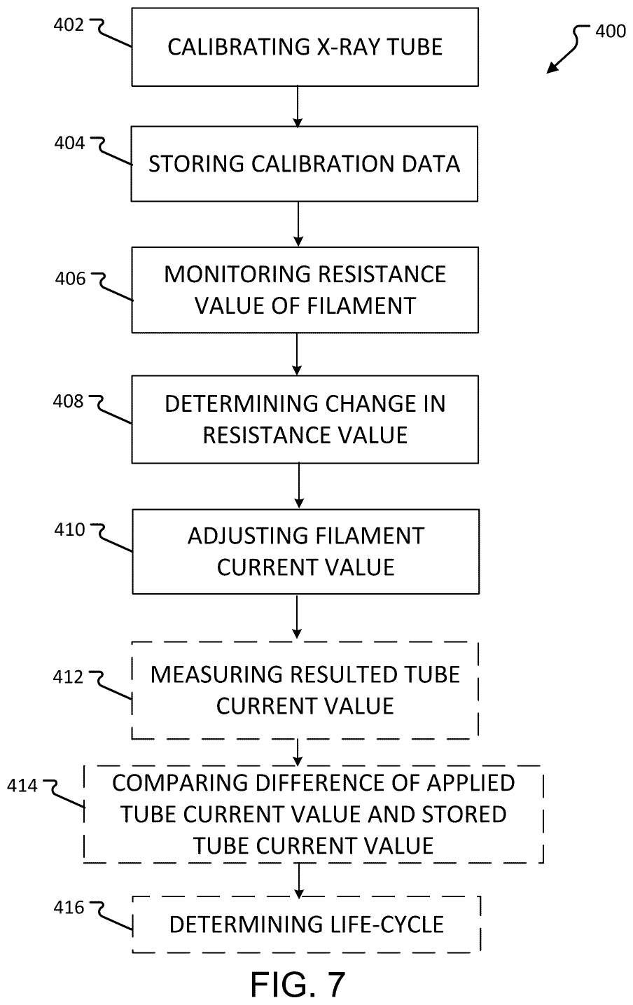

FIG. 7 depicts a flowchart illustrating a method 400 of adaptively controlling filament current in an x-ray tube. The example methods and operations can be implemented or performed by the systems and devices described herein (e.g., the imaging system 100 and x-ray tube 200 shown in FIGS. 1-3). The method 400 begins with calibrating (or re-calibrating) the x-ray tube having a filament therein (operation 402) and storing the calibration data at the imaging system (operation 404). In an aspect, the calibration data includes a filament current value that determines a tube output value for a tube voltage value at a plurality of stations that is generated from the calibration of the x-ray tube. Each station is defined by the tube output value in mA and the tube voltage value in kV so that a required or desired x-ray radiation amount is generated by the x-ray tube. In some examples, the filament current value may include a filament voltage value that is applied across the filament to generate the current value. The x-ray calibration procedure can be performed by methods that are currently known (e.g., a substitution method) or developed in the future. Because every x-ray tube has slightly different performance characteristics, calibration enables accurate operation of the imaging system as described herein.

The method 400 continues with monitoring a resistance value of the filament over a period of time (operation 406). As described above, the filament material evaporates under high temperatures and results in a thinning of the filament material, thereby increasing filament resistance over the operational lifetime of the x-ray tube. In an aspect, monitoring the resistance value of the filament may include measuring the resistance value after a predetermined number of sequence exposures and storing each measured resistance value. Based on the stored resistance values a resistance curve can be generated. In an example, the period of time for monitoring the resistance value can be based on the number of exposure counts and for example resistance values can be measured every 100 sequence exposures since it is known that resistance changes in an order many times this predetermined number. In other examples, the resistance value can be measured after every sequence exposure and stored as required or desired. As such, measuring and storing the resistance value is performed periodically.

In the example, measuring the resistance value includes applying a constant and known filament current value to the filament so as to measure the resistance. In an aspect, filament voltage is applied so that the filament receives about 2.5 amps to measure the resistance since voltage, current, and resistance are related via Ohm's law. Because each sequence exposure generates heat at the filament, in some examples, prior to measuring the resistance value, the filament is allowed to cool so as to increase accuracy of the resistance value measurement. In an example, cooling the filament can include waiting a predetermined time period between an end of the sequence exposures and prior to applying the constant filament current value for measuring the resistance value of the filament.

Turning back to the method 400, once the resistance value of the filament is being monitored (operation 406), a change in the resistance value of the filament is determined over the period of time (operation 408). Because the resistive characteristics of the filament change slowly after every exposure, and the tolerance for each exposure does allow for some change to the tube current output, the filament current value does not need to be revised or adjusted for every exposure count. As such, the change in resistance value can be determined by comparing a difference between resistance values over a period of time to a predetermined benchmark. For example, the predetermined benchmark may be a 4% resistance value change over at least 10,000 exposure counts. It should be appreciated that other predetermined benchmark values are also contemplated herein. For example, but not limiting, the resistance value change may be 2%, 5%, 10%, or the like, and the exposure count may be 5,000, 8,000, 12,000, or the like as required or desired.

Once it has been determined that the resistance value of the filament has changed (operation 408), then the filament current value is adjusted for at least one of the plurality of stations based on the changed resistance value (operation 410). By adjusting the filament current applied at the filament, the power at the filament during operation can be the same as the calibrated data for the tube current value and the tube voltage value for the station. Accordingly, the x-ray tube can deliver the required or desired tube current value even with an ever changing filament resistance value. In an aspect, the filament current value of each of the plurality of stations is updated based on the changed resistance value. As such, the calibration data can be adjusted based on the current resistive properties of the filament, and less active stations are still configured for a more accurate tube current value output the next time those stations are used for imaging procedures. In an aspect, adjusting the filament current value is based on a power model, whereby the filament power is maintained as right after initial filament calibration.

In the example, once the filament current value is adjusted the process may repeat itself with monitoring the resistance value over a new and reset period of time, determining change in the resistance value, and further adjust the filament current value as required or desired. This feedback algorithm for the resistance value enables the imaging system to operate longer between maintenance operations that calibrate/re-calibrate the x-ray tube.

In aspects, the method 400 may further include measuring a resulted tube current value from the x-ray tube at a sequence exposure having a stored tube current value and tube voltage value for a station of the plurality of stations (operation 412), and comparing a difference between the measured resulted tube current value and the stored tube current value for the respective station (operation 414). By measuring the resulted mA tube output current from the x-ray tube during operation and comparing the measured value to the stored desired output, operational data of the x-ray tube may be generated. For example, the accuracy of the feedback algorithm can be verified by this operational data, and if the adjusted filament current value results in less accurate tube current output values, a new filament calibration may be required. In another example, if the difference between the measured tube output current and the desired output is higher than a predetermined threshold, a new filament calibration may be required.

In other examples, based on the difference between the measured resulted tube current value and the stored tube current value, the period of time is at least partially defined. For example, instead of monitoring the resistance value based on the number of exposure counts, the resistance value can be monitored based on the operational results of the tube head, and if the difference between the measured tube current and the stored desired tube current exceeds a predetermined threshold, the change in resistance value can be used to adjust the filament current value as described above. In aspects, the measurement of the resulted tube current value may be performed pre-exposure or post-exposure of the sequence exposure. For example, in tomosynthesis imaging modalities the tube current measurements may be performed post-exposure, while in mammography imaging modalities the tube current measurement may be performed pre-exposure.

The method 400 may also include using the monitored resistance value of the filament to determine a life-cycle period of the filament (operation 416). For example, because filament resistance increases during the operational life of the filament due to filament material evaporating, an upper limit of filament resistance can be defined that relates to the filament material being too thin and triggering a tube replacement and a new filament calibration for the new x-ray tube.

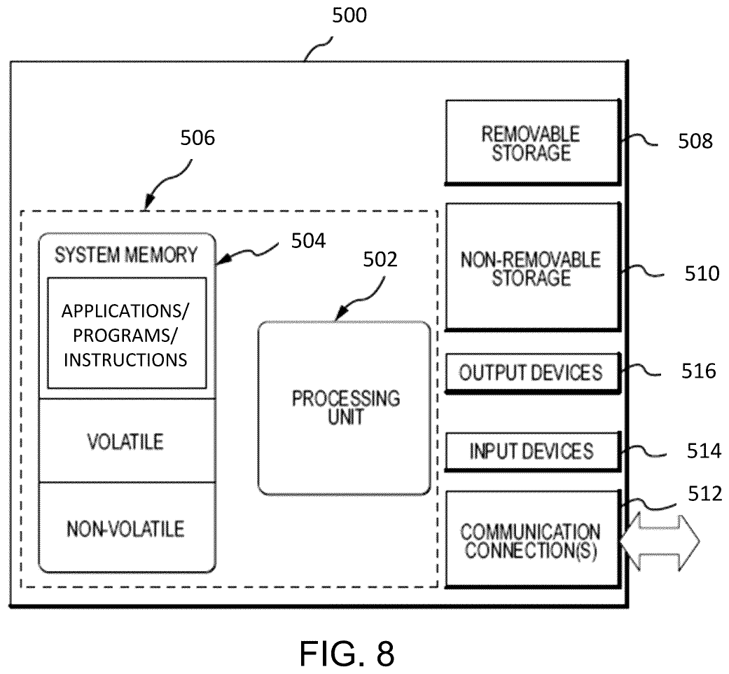

FIG. 8 illustrates an exemplary suitable operating environment 500 for controlling an x-ray tube. This operating environment may be incorporated directly into the high voltage control circuit disclosed herein, or may be incorporated into a computer system discrete from, but used to control the breast imaging systems described herein. Such computer system may be, for example, the system control and work station 136 depicted in FIG. 1. This is only one example of a suitable operating environment and is not intended to suggest any limitation as to the scope of use or functionality. Other well-known computing systems, environments, and/or configurations that can be suitable for use include, but are not limited to, imaging systems, personal computers, server computers, hand-held or laptop devices, multiprocessor systems, microprocessor-based systems, programmable consumer electronics such as smart phones, network PCs, minicomputers, mainframe computers, tablets, distributed computing environments that include any of the above systems or devices, and the like.

In its most basic configuration, operating environment 500 typically includes at least one processing unit 502 and memory 504. Depending on the exact configuration and type of computing device, memory 504 (storing, among other things, instructions to read from data storage devices or sensors, or perform other methods disclosed herein) can be volatile (such as RAM), non-volatile (such as ROM, flash memory, etc.), or some combination of the two. This most basic configuration is illustrated in FIG. 8 by dashed line 506. Further, environment 500 can also include storage devices (removable, 508, and/or non-removable, 510) including, but not limited to, magnetic or optical disks or tape. Similarly, environment 500 can also have input device(s) 514 such as touch screens, keyboard, mouse, pen, voice input, etc., and/or output device(s) 516 such as a display, speakers, printer, etc. Also included in the environment can be one or more communication connections 512, such as LAN, WAN, point to point, Bluetooth, RF, etc. In embodiments, the connections may be operable to facility point-to-point communications, connection-oriented communications, connectionless communications, etc.

Operating environment 500 typically includes at least some form of computer readable media. Computer readable media can be any available media that can be accessed by processing unit 502 or other devices having the operating environment. By way of example, and not limitation, computer readable media can include computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, solid state storage, or any other tangible medium which can be used to store the desired information. Computer storage media does not include communication media.

Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media. A computer-readable device is a hardware device incorporating computer storage media.

The operating environment 500 can be a single computer operating in a networked environment using logical connections to one or more remote computers. The remote computer can be a personal computer, a server, a router, a network PC, a peer device or other common network node, and typically includes many or all of the elements described above as well as others not so mentioned. The logical connections can include any method supported by available communications media. Such networking environments are commonplace in offices, enterprise-wide computer networks, intranets and the Internet.

The embodiments described herein may be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices may be employed to perform the functionality disclosed herein without departing from the scope of the disclosure. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

This disclosure describes some examples of the present technology with reference to the accompanying drawings, in which only some of the possible examples were shown. Other aspects may, however, be embodied in many different forms and should not be construed as limited to the examples set forth herein. Rather, these examples were provided so that this disclosure was thorough and complete and fully conveyed the scope of the possible examples to those skilled in the art. Further, as used herein and in the claims, the phrase “at least one of element A, element B, or element C” is intended to convey any of: element A, element B, element C, elements A and B, elements A and C, elements B and C, and elements A, B, and C. Additionally, one having skill in the art will understand the degree to which terms such as “about” or “substantially” convey in light of the measurement techniques utilized herein. To the extend such terms may not be clearly defined or understood by one having skill in the art, the term “about” shall mean plus or minus ten percent.

Although specific examples were described herein, the scope of the technology is not limited to those specific examples. One skilled in the art will recognize other examples or improvements that are within the scope and spirit of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

1. (canceled)

2. A method of controlling an x-ray tube of an imaging system comprising:
receiving, at the imaging system, calibration data for the x-ray tube, the calibration data including a filament current value for a plurality of exposure values based on different tube current and tube voltage combinations;
storing the calibration data at the imaging system;
based on the stored calibration data, controlling the x-ray tube, via a high voltage control circuit of the imaging system operationally coupled to the x-ray tube, to emit a plurality of sequence exposures;
measuring a resistance value, via the high voltage control circuit, of a filament of the x-ray tube after a predetermined number of sequence exposures of the plurality of sequence exposures;
storing each measured resistance value at the imaging system;
determining, at the imaging system, a change in the stored measured resistance values of the filament, wherein the determined changed resistance value is based on a difference between the stored measured resistance values over a number count of the emitted plurality of sequence exposures; and
adjusting the filament current value within the calibration data stored at the imaging system for at least one of the plurality of exposure values based on the determined changed resistance value.

3. The method of claim 2, wherein measuring the resistance value includes applying, via the high voltage control circuit, a constant filament current value to the filament after each of the predetermined number of sequence exposures.

4. The method of claim 3, further comprising waiting a predetermined time between an end of a sequence exposure and the application of the constant filament current value.

5. The method of claim 3, wherein the application of the constant filament current value is performed when the filament has cooled after a sequence exposure.

6. The method of claim 2, wherein the plurality of sequence exposures of the imaging system includes mammography exposures and tomosynthesis exposures.

7. The method of claim 2, further comprising:
measuring, via the high voltage control circuit, a tube exposure output of the x-ray tube of at least one of the plurality of sequence exposures;
calculating a difference between the measured tube exposure output and a desired tube exposure output based on the stored calibration data; and
adjusting a filament voltage value within the high voltage control circuit when the calculated difference is within a predetermined value.

8. The method of claim 7, further comprising alerting, via the imaging system, for a new x-ray tube calibration when the calculated difference is outside of the predetermined value.

9. The method of claim 7, wherein the measurement of the tube exposure output is pre-exposure during a mammography exposure.

10. The method of claim 7, wherein the measurement of the tube exposure output is post-exposure during a tomosynthesis exposure.

11. The method of claim 2, wherein the adjustment of the filament current value within the calibration data is based on a predetermined benchmark value.

12. The method of claim 11, wherein the predetermined benchmark value is at least a resistance value change of the filament of at least 2% over at least 5,000 emitted sequence exposures.

13. The method of claim 11, wherein the predetermined benchmark value is a measured tube exposure output that is at least 20% different than a desired tube exposure output based on the stored calibration data.

14. The method of claim 2, further comprising alerting, via the imaging system, for a replacement x-ray tube when the measured resistance value approaches an upper limit resistance value.

15. The method of claim 2, wherein the adjustment of the filament current value within the calibration data occurs for each of the plurality of exposure values based on the determined changed resistance value.

16. An imaging system comprising:
an x-ray tube having a filament;
a high voltage control circuit operationally coupled to the x-ray tube;
at least one processor communicatively coupled to the high voltage control circuit; and
memory communicatively coupled to the at least one processor, the memory comprising computer executable instructions configured to:
receive calibration data for the x-ray tube, the calibration data including a filament current value for a plurality of exposure values based on different tube current and tube voltage combinations;
store the calibration data;
based on the stored calibration data, control the x-ray tube to emit a plurality of sequence exposures;
measure a resistance value of the filament of the x-ray tube after a predetermined number of sequence exposures of the plurality of sequence exposures;
store each measured resistance value;
determine a change in the stored measured resistance values of the filament, wherein the determined changed resistance value is based on a difference between the stored measured resistance values over a number count of the emitted plurality of sequence exposures; and
adjust the filament current value within the stored calibration data for at least one of the plurality of exposure values based on the determined changed resistance value.

17. The imaging system of claim 16, wherein the imaging system is a multi-modal imaging system and includes a mammography mode and a tomosynthesis mode.

18. The imaging system of claim 16, wherein the high voltage control circuit includes a monitoring instrument configured to measure one or more electrical properties of the filament.

19. The imaging system of claim 16, wherein when the measured resistance value approaches an upper limit resistance value, the imaging system is configured to alert for a replacement x-ray tube.

20. The imaging system of claim 16, wherein the computer executable instructions are further configured to:
measure a tube exposure output of the x-ray tube of at least one of the plurality of sequence exposures;
calculate a difference between the measured tube exposure output and a desired tube exposure output based on the stored calibration data; and
alert for a new x-ray tube calibration when the calculated difference is outside of a predetermined value.

21. The imaging system of claim 16, wherein the measurement of the resistance value occurs after the filament has cooled after a sequence exposure.
